(19) United States
(12) Patent Application Publication
(10) Pub. No.: US 2014/0046277 A1
(43) Pub. Date: Feb. 13, 2014

(54) FAST-CLOTTING WOUND DRESSINGS

(71) Applicant: David Henry Farrell, Tualatin, OR (US)

(72) Inventor: David Henry Farrell, Tualatin, OR (US)

(73) Assignee: GAMMA THERAPEUTICS, INC.

(21) Appl. No.: 14/112,013

(22) PCT Filed: Apr. 12, 2012
(86) PCT No.: PCT/US12/33364

§ 371 Date: Oct. 15, 2013

Related U.S. Application Data
(60) Provisional application No. 61/489,510, filed on May 24, 2011.
(60) Provisional application No. 61/476,172, filed on Apr. 15, 2011.

(51) Int. Cl. A61L 15/32; A61L 15/44

(52) U.S. Cl. CPC A61L 15/32; A61L 15/44
USPC 604/304

(57) ABSTRACT

Disclosed herein are fast-clotting wound dressings impregnated with one or more hemostatic agents including, for example, tissue factor, and optionally, one or more phospholipids. Such dressings may be used to initiate blood clotting on a wound of a subject.

### FIELD OF THE DISCLOSURE

The present disclosure provides fast-clotting wound dressings impregnated with one or more hemostatic agents (e.g. tissue factor) and optionally one or more phospholipids.

### BACKGROUND OF THE DISCLOSURE

Trauma is the leading cause of death for all persons between the ages of 1 and 44 years. The majority of preventable deaths that occur following a traumatic event are the result of hemorrhage, both in civilians and in military combat personnel.

Seven ideal qualities for wound dressings have been identified [Pusateri et al., J Trauma. 2003; 54:177-82]. These include that the dressing should be (1) able to stop large-vessel arterial and venous bleeding within 2 minutes of application on the wound, even when applied to an actively bleeding site through a pool of blood (a likely field scenario); (2) ready to use, with no requirement for mixing or special preparation; (3) simple to apply by the wounded individual, his buddy, or a medic/emergency medical technician with minimal training required; (4) lightweight and durable; (5) stable and functional at room temperature for at least 2 years and in extreme ambient temperatures (between −10° C. and 55° C.) for several weeks or longer; (6) safe to use, posing no risk of either injury in the tissue to which it is applied or of bacterial or viral transmission and (7) inexpensive.

Current wound dressings to stop hemorrhage suffer drawbacks including that clotting times are not fast enough; application of the dressings can cause thermal damage and/or pain in the area of the injury; agents incorporated into the dressings can increase the risk of thrombotic events and/or the dressings are not pliable enough or appropriate for use with large and/or irregularly shaped wounds. Moreover, there is currently not a dressing available that meets all 7 of the characteristics noted above.

The Committee on Tactical Combat Casualty does not currently recommend hemostatic dressings for use in care under fire scenarios. Based on the foregoing, there is room for significant improvement in wound dressings used to stop hemorrhage.

### SUMMARY OF THE DISCLOSURE

The present disclosure relates generally to wound dressings comprising one or more hemostatic agents such as tissue factor (used interchangeably with TF herein) and optionally comprising one or more phospholipids.

The present disclosure provides a fast-clotting wound dressing impregnated with a hemostatic agent such as tissue factor and/or analogues, homologues, precursors or derivatives thereof.

The present disclosure also provides a fast-clotting wound dressing impregnated with a hemostatic agent (e.g., tissue factor) and a phospholipid.

In an embodiment, the hemostatic agent is tissue factor including, for example, human tissue factor. In a further embodiment, the human tissue factor is produced using recombinant technology.

In an embodiment, the wound dressing initiates clotting within 120, 100, 80, 60, 50, 40, 35, 30, 27, 26, 25, 24, 23, 22, 21, 20, 19, 18, 17, 16, 15, 14, 13, 12, 11, 10, 9, 8, 7, 6, 5, 4, 3, 2, or 1 second(s) of application to a wound.

In an embodiment, the wound dressing initiates clotting within 27 seconds of application to a wound.

In an embodiment, the hemostatic agent (e.g., tissue factor) is impregnated in the dressing at a concentration of 5 pmoles of recombinant tissue factor per bandage, 10 pmoles (0.34 μg) of recombinant tissue factor per bandage, 20 pmoles per bandage, 30 pmoles per bandage, 40 pmoles per bandage, 50 pmoles per bandage, 60 pmoles per bandage, 70 pmoles per bandage, 80 pmoles per bandage, 90 pmoles per bandage, or 100 pmoles per bandage.

In an embodiment, the dressing is further impregnated with a secondary clotting factor. In an embodiment, the dressing is further impregnated with pharmaceuticals, antifungal agents, topical pain reducing medication, pharmaceuticals, anti-inflammatory agents and/or tissue enzyme inhibitors. In further embodiments, the pharmaceutical is an analgesic, an anti-inflammatory agent, or an antibacterial.

The present disclosure also provides methods for treating an individual having a wound where blood is present comprising applying a fast-clotting wound dressing as provided herein to the wound on the individual.

In an embodiment, the individual has an acquired coagulopathy.

In an embodiment, the individual has a traumatic injury which causes bleeding.

In an embodiment, the individual is undergoing a surgical procedure.

The present disclosure also provides methods to initiate blood clotting on a wound of a subject in need thereof comprising applying a fast-clotting wound dressing impregnated with a hemostatic agent (e.g., tissue factor and/or analogues, homologues, precursors or derivatives thereof) to the wound.

The present disclosure also provides methods to initiate blood clotting on a wound of a subject in need thereof comprising applying a fast-clotting wound dressing impregnated with a hemostatic agent (e.g., tissue factor and/or analogues, homologues, precursors or derivatives thereof) and a phospholipid to the wound.

In an embodiment, the hemostatic agent is tissue factor such as human tissue factor. In a further embodiment, the human tissue factor is recombinantly produced.

In an embodiment, the wound dressing initiates clotting within 120, 100, 80, 60, 50, 40, 35, 30, 27, 26, 25, 24, 23, 22, 21, 20, 19, 18, 17, 16, 15, 14, 13, 12, 11, 10, 9, 8, 7, 6, 5, 4, 3, 2, or 1 second(s) of application to a wound.

In an embodiment, the wound dressing initiates clotting within 27 seconds of application to a wound.

In an embodiment, the hemostatic agent (e.g., tissue factor) is impregnated in the dressing at a concentration of 5 pmoles of recombinant tissue factor per bandage, 10 pmoles (0.34 μg) of recombinant tissue factor per bandage, 20 pmoles per bandage, 30 pmoles per bandage, 40 pmoles per bandage, 50 pmoles per bandage, 60 pmoles per bandage, 70 pmoles per bandage, 80 pmoles per bandage, 90 pmoles per bandage, or 100 pmoles per bandage.

In an embodiment, the dressing is further impregnated with a secondary clotting factor.

In an embodiment, the dressing is further impregnated with pharmaceuticals, antifungal agents, topical pain reducing medication, pharmaceuticals, anti-inflammatory agents and/or tissue enzyme inhibitors. In a further embodiment, the pharmaceutical is an analgesic, an anti-inflammatory agent, or an antibacterial.

In an embodiment, the wound comprises broken skin tissue.

In an embodiment, the wound comprises broken soft tissue within a human body.

The present disclosure also provides a kit for initiating blood clotting, the kit comprising a fast-clotting wound dressing impregnated with a hemostatic agent (e.g., tissue factor and/or analogues, homologues, precursors or derivatives thereof); and instructions for using the kit.

The present disclosure also provides a kit for initiating blood clotting, the kit comprising a fast-clotting wound dressing impregnated with a hemostatic agent (e.g., tissue factor and/or analogues, homologues, precursors or derivatives thereof); a phospholipid; and instructions for using the kit.

The present disclosure also provides a fast-clotting wound dressing impregnated with one or more hemostatic agents and one or more phospholipids. In an embodiment, the hemostatic agent is tissue factor and/or analogues, homologues, precursors or derivatives thereof. In another embodiment, the tissue factor is human tissue factor. In another embodiment, the human tissue factor is produced using recombinant technology.

In another embodiment, the one or more hemostatic agents are tissue factor, chitosan, deacetylated chitin, zeolite, fibrin sealant, smectite, iron oxyacid salt, kaolin, celite, micronized silica and/or ellagic acid.

In another embodiment, the one or more phospholipids are phosphatidylcholine, phosphatidylserine and/or sphingomyelin or derivatives thereof.

In another embodiment, the wound dressing initiates clotting within 120 seconds of application to a wound. In another embodiment, the wound dressing initiates clotting within 27 seconds of application to a wound.

In another embodiment, the hemostatic agent is impregnated at a concentration of 5 pmoles-100 pmoles per bandage. In another embodiment, the phospholipid is impregnated at a concentration of 5 pmoles-100 pmoles per bandage.

In another embodiment, the dressing is further impregnated with an additional clotting factor. In another embodiment, the dressing is further impregnated with one or more agents selected from the group consisting of pharmaceuticals, antifungal agents, topical pain reducing medication, pharmaceuticals, anti-inflammatory agents and tissue enzyme inhibitors. In another embodiment, the pharmaceutical is an analgesic, an anti-inflammatory agent, or an antibacterial.

Additional methods disclosed herein include methods for treating an individual having a wound where bleeding is present comprising applying a fast-clotting wound dressing described above or elsewhere herein to the wound on the individual.

In another embodiment, the individual has an acquired coagulopathy. In another embodiment, the individual has a traumatic injury which causes the bleeding. In another embodiment, the individual is undergoing a surgical procedure.

Methods disclosed herein further include methods to initiate blood clotting on a wound of a subject in need thereof comprising applying a fast-clotting wound dressing impregnated with a hemostatic agent and a phospholipid to the wound.

In exemplary embodiments of these methods, the hemostatic agent can be tissue factor, chitosan, deacetylated chitin, zeolite, fibrin sealant, smectite, iron oxyacid salt, kaolin, celite, micronized silica and/or ellagic acid.

In further embodiments of these methods, the wound dressings initiate clotting within 1-27 second(s) of application to a wound.

In another embodiment, hemostatic agents and/or phospholipids can be impregnated at a concentration of 5 pmoles-100 pmoles per bandage.

In another embodiment, additional clotting factors can be incorporated into the dressing. Additionally, or alternatively, the dressings can be further impregnated with one or more agents selected from the group consisting of pharmaceuticals, antifungal agents, topical pain reducing medication, pharmaceuticals, anti-inflammatory agents and/or tissue enzyme inhibitors. In an embodiment disclosed herein, the additionally incorporated agent is an analgesic, an anti-inflammatory agent, or an antibacterial.

In an embodiment of the methods disclosed herein, the wound comprises broken skin tissue. In another embodiment, the wound comprises broken soft tissue within a human body.

In certain embodiments of the methods described above, the one or more phospholipids are phosphatidylcholine, phosphatidylserine and/or sphingomyelin or derivatives thereof.

Embodiments disclosed herein also include kits. In an embodiment of the kits, the kit is provided with components that initiate blood clotting which include a fast-clotting wound dressing impregnated with a hemostatic agent and a phospholipid; and instructions for using the kit.

Kits described herein can also include any dressing described above or elsewhere within the current disclosure and provide instructions to practice any method disclosed above or elsewhere herein.

In a particular embodiment, the kit includes a fast-clotting wound dressing impregnated with one or more of tissue factor, chitosan, deacetylated chitin, zeolite, fibrin sealant, smectite, iron oxyacid salt, kaolin, celite, micronized silica and/or ellagic acid co-impregnated with a phospholipid. In another embodiment, the phospholipid is phosphatidylcholine, phosphatidylserine and/or sphingomyelin or derivatives thereof.

### BRIEF DESCRIPTION OF THE FIGURES

The foregoing summary, as well as the following detailed description of the disclosure, will be better understood when read in conjunction with the appended figures. The described figures are provided to further illustrate the disclosure. It should be understood, however, that the disclosure is not limited to the precise arrangements, examples and instrumentalities shown. Additional features and advantages are described herein, and will be apparent from disclosure herein.

FIG. 1 shows coagulation initiation pathways through either intrinsic or extrinsic pathways.

FIG. 2 shows coagulation times of normal human plasma at 37° C. induced by different wound dressings.

FIG. 3 shows coagulation times of a wound dressing impregnated with TF and a phospholipid (GammaTF) versus coagulation times of Combat Gauze® (Z-Medica Corporation, Wallingford, Conn.).

FIG. 4 shows reference sequences for TF and nucleotide sequences encoding TF.

### DETAILED DESCRIPTION

Current wound dressings to stop hemorrhage suffer drawbacks including that clotting times are not fast enough; application of the dressings can cause thermal damage and/or pain in the area of the injury; agents incorporated into the dressings can increase the risk of thrombotic events and/or the dressings are not pliable enough or appropriate for use with large and/or irregularly shaped wounds.

Disclosed herein are fast-clotting wound dressings that address these drawbacks among others. The wound dressings disclosed herein are impregnated with one or more hemostatic agents including, for example, tissue factor and/or analogues, homologues, precursors or derivatives thereof (collectively TF). Optionally the wound dressing can also be impregnated with one or more phospholipids. Such addition of a phospholipid as a co-factor to a hemostatic agent has been found to reduce clotting time even further than that achieved with a hemostatic agent alone. Also disclosed are uses of TF to treat a variety of other disorders, conditions or events associated with unwanted or uncontrolled bleeding.

In certain embodiments, wound dressings as disclosed herein can unexpectedly initiate clotting within 120, 100, 80, 60, 50, 40, 35, 30, 27, 26, 25, 24, 23, 22, 21, 20, 19, 18, 17, 16, 15, 14, 13, 12, 11, 10, 9, 8, 7, 6, 5, 4, 3, 2, or 1 second(s) of application to a wound. In another embodiment, wound dressings as disclosed herein can unexpectedly initiate clotting within 27 seconds or less; within 26 seconds or less; within 25 seconds or less; within 24 seconds or less; within 23 seconds or less; within 22 seconds or less; within 21 seconds or less; within 20 seconds or less; within 19 seconds or less; within 18 seconds or less; within 17 seconds or less; within 16 seconds or less; within 15 seconds or less; within 14 seconds or less; within 13 seconds or less; within 12 seconds or less; within 11 seconds or less; within 10 seconds or less; within 9 seconds or less; within 8 seconds or less; within 7 seconds or less; within 6 seconds or less; within 5 seconds or less; within 4 seconds or less; within 3 seconds or less; within 2 seconds or less; within 1 second or less; and/or upon contact with a wound to be treated. In particular embodiments, clotting time can be measured using, without limitation, the activated partial thromboplastin assay (aPTT), whole blood prothrombin time (PT), the activated clotting time (ACT), the recalcified activated clotting time, or the Lee-White Clotting time.

Hemostatic agents and phospholipids can be placed on the wound dressings disclosed herein in amounts of 5 pmoles per bandage, 10 pmoles (0.34 μg) per bandage, 20 pmoles per bandage, 30 pmoles per bandage, 40 pmoles per bandage, 50 pmoles per bandage, 60 pmoles per bandage, 70 pmoles per bandage, 80 pmoles per bandage, 90 pmoles per bandage, or 100 pmoles per bandage or any amount in between 5 pmoles per bandage and 100 pmoles per bandage. The preceding amounts can be per hemostatic agent and per phospholipid or account for all hemostatic agents and phospholipids combined on a dressing.

Hemostatic agents and phospholipids can be incorporated onto the dressings disclosed herein in the following non-limiting ratios of hemostatic agent to phospholipid: 0.001:1 or lower to 2:1 or higher; 0.05:1 or lower to 2:1 or higher or 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to 1:1, 1.1:1, 1.2:1, 1.3:1, 1.4:1, 1.5:1; 3:1, 4:1 or 5:1. In other embodiments, higher or lower ratios can be used.

Tissue factor as a single hemostatic agent or in combination with other hemostatic agents can be employed. Useful loading levels for the hemostatic agent(s) on a hemostatic material can vary, depending upon, for example, the nature of the selected material, the number of hemostatic agent(s), the form of the material, and the nature of the wound to be treated. However, in general in the case of hemostatic gauze, a generally useful weight ratio of hemostatic agent to hemostatic gauze is from 0.001:1 or lower to 2:1 or higher. In certain embodiments, a weight ratio of hemostatic agent(s) to hemostatic material can be from 0.05:1 or lower to 2:1 or higher. More preferably, a weight ratio from 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to 1:1, 1.1:1, 1.2:1, 1.3:1, 1.4:1, or 1.5:1 can also be employed. In other embodiments, higher or lower ratios can be used.

When phospholipids are utilized as well, phospholipids can similarly be included in the generally useful weight ratios of phospholipid to hemostatic gauze at from 0.001:1 or lower to 2:1 or higher. In certain embodiments, a weight ratio of phoshpholipid to hemostatic material can be from 0.05:1 or lower to 2:1 or higher. More preferably, a weight ratio from 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to 1:1, 1.1:1, 1.2:1, 1.3:1, 1.4:1, or 1.5:1 can also be employed. In other embodiments, higher or lower ratios can be used.

The preceding ratios can be applied to hemostatic agent(s) and phospholipid(s) individually or in combination when applied to a particular hemostatic material, such as gauze.

Wound dressings disclosed herein can be used to initiate clotting on all wound types including, without limitation, large wounds; deep wounds, irregular wounds; wounds on the head, scalp, neck, torso, chest, back, abdomen, groin, wounds on the extremities, wounds to soft tissue, etc. Wound dressings disclosed herein can also be appropriate for intracorporal use, for example, to initiate clotting during a surgical procedure. Relevant non-limiting surgical procedures include, without limitation, heart surgery, cardiac valve surgery, aortic valve replacement, angioplasty, lung surgery, abdominal surgery, spinal surgery, brain surgery, vascular surgery, dental surgery, cancer surgery, surgery on duodenal ulcers, resection of recurrent hemangiopericytoma or organ transplant surgery, including transplantation of bone marrow, heart, lung, pancreas or liver. Accordingly, the wound dressing disclosed herein can be used to initiate clotting in, without limitation, the skin, brain, inner ear region, eyes, liver, lung, tumor tissue, gastrointestinal tract, etc.

Wounds treated by embodiments disclosed herein can be caused by, without limitation trauma or traumatic injury (used interchangeably herein). Trauma is defined as an injury to living tissue by an extrinsic agent. Trauma is classified as either blunt trauma (resulting in internal compression, organ damage and internal hemorrhage) or penetrative trauma (a consequence of an agent penetrating the body and destroying tissue, vessel and organs, resulting in external hemorrhaging). Trauma can be caused by several events including, but not limited to, vehicle accidents (causing blunt and/or penetrative trauma), gun shot wounds (causing penetrative trauma), stabbing wounds (causing penetrative trauma), machinery accidents (causing penetrative and/or blunt trauma), and falls from significant heights (causing penetrative and/or blunt trauma).

While embodiments disclosed thus far have focused on bleeding due to trauma and/or surgical intervention in individuals with otherwise normal coagulation systems, the dressings disclosed herein can also be useful to stop unwanted or uncontrolled bleeding in individuals with blood clotting deficiencies. For example, tissue factor inhibitors can develop spontaneously in otherwise healthy individuals. This can result in life-threatening hemorrhage. Common sites of bleeding are skin, mucosa, muscles and retroperitoneum. This bleeding can be reduced with the wound dressings impregnated with one or more hemostatic agents (e.g., tissue factor) and optionally one or more phospholipids as provided herein.

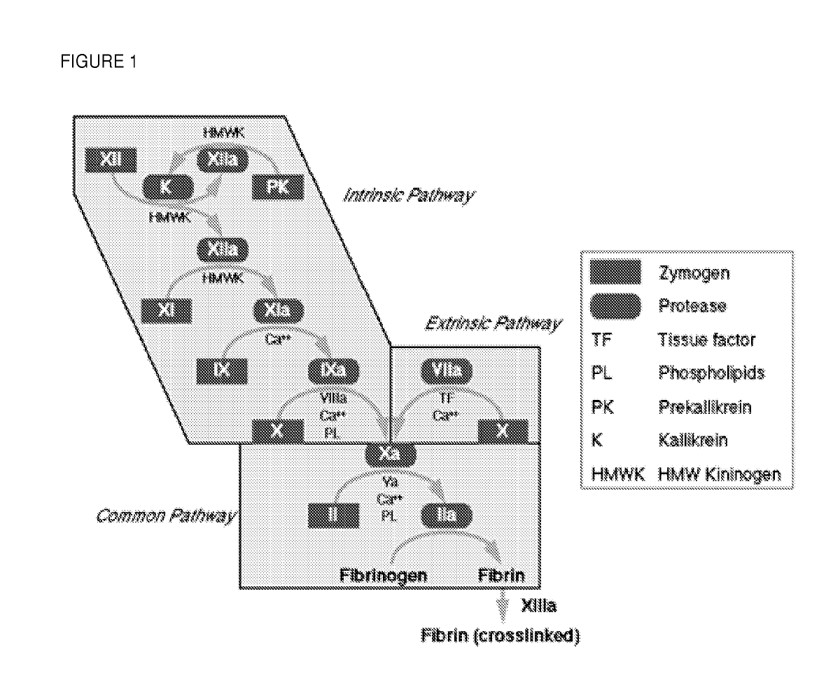

The effects of coagulation disorders not specifically related to TF can also be reduced or eliminated with the dressings disclosed herein. Without being bound by theory or mechanism of action, TF-impregnated wound dressings can control bleeding in patients with defective coagulation systems by driving clotting mechanisms to the extrinsic clotting pathway, by-passing the intrinsic clotting pathway where a number of clotting disorders arise (see FIG. 1). Accordingly, the wound dressings disclosed herein can be used to initiate clotting in individuals with blood coagulation disorders, hematologic disorders, hemorrhagic disorders, hemophiliac (congenital or acquired), such as hemophilia A, hemophilia B, factor V deficiency, factor VII deficiency, factor X deficiency and acquired blood disorders, such as acquired factor VII deficiency caused by liver disease. Wound dressings disclosed herein can also be used in the treatment of additional bleeding diseases and disorders, such as, but not limited to, thrombocytopenia (e.g., such as due to chemotherapeutic regimes), Von Willebrand's disease, hereditary platelet disorders (e.g., storage pool disease such as Chediak-Higashi and Hermansky-Pudlak syndromes, thromboxane A2 dysfunction, Glanzmann's thrombasthenia, and Bernard-Soulier syndrome), hemolytic-uremic syndrome, Hereditary Hemorrhagic Telangiectsia, also known as Rendu-Osler-Weber syndrome, allergic purpura (Henoch Schonlein purpura) and disseminated intravascular coagulation.

Relevant coagulopathies can also result from conditions including, but not limited to, fulminant hepatic failure (FHF; such as caused by hepatoxic drugs, toxins, metabolic diseases, infectious diseases and ischemia), other liver disease, including cirrhosis and disease associated with Wilson's disease, vitamin K deficiency (such as caused by antibiotic treatment or diet), hemolytic uremic syndrome, thrombotic thrombocytopenia (TTC) and disseminated intravascular coagulopathy (DIC).

As used herein, “dressing” and “bandage” may be used interchangeably to refer to a device that can be used to cover, dress, protect, or heal a wound. As used herein, a “wound” includes damage to any tissue in a living organism. The tissue can be internal, external, or a combination thereof. The tissue can be hard or soft tissue. The wound can include any lesion resulting from an agent, injury, disease, infection, or surgical intervention. Dressings or bandages (e.g., materials capable of promoting hemostasis, termed herein a “hemostatic material”) of the present disclosure comprise one or more hemostatic agents (e.g., TF) in a therapeutic amount as the sole active compounds or in combination with one or more additional active compounds or phospholipids. As used herein, a “therapeutic amount” of a hemostatic agent is an amount that promotes blood coagulation, clot formation, or both. One of ordinary skill in the art can readily determine the optimal therapeutic amount of a hemostatic agent with routine methods known in the art.

As used herein, “hemostatic agent” may refer to a substance that promotes hemostasis and blood clotting. Exemplary hemostatic agents include, without limitation, natural or recombinant blood coagulation factors, chitosan, biodegradable, nontoxic, deacetylated forms of chitin (HemCon®, HemCon Medical Technologies, Inc., Tigard, Oreg. and Celox®, MedTrade Products, Ltd., Cheshire, UK); zeolite, a non-biodegradable oxide of silicon, aluminum, sodium, magnesium & quartz (QuikClot®, Z-Medica Corporation, Wallingford, Conn.); fibrin sealant dressing, a biodegradable natural clotting factor initiated with thrombin (FSD); smectite, a non-biodegradable nonmetallic clay mineral of sodium, calcium and aluminum silicate (WoundStat®, TraumaCure, Inc., Bethesda, Md.], and a non-biodegradable iron oxyacid salt with a hydrophilic polymer. Additional hemostatic agents include, without limitation, kaolin, celite, micronized silica, or ellagic acid. The clotting times of bandages impregnated with tissue factor and/or these hemostatic agents can be improved by co-impregnation with one or more phospholipids. In particular embodiments, the phospholipids can be phosphatidylcholine, phosphatidylserine and/or sphingomyelin.

As used herein, “coagulation pathway” or “coagulation cascade” may refer to the series of activation events that leads to the formation of an insoluble fibrin clot. In the coagulation cascade or pathway, an inactive protein of a serine protease (also called a zymogen) is converted to an active protease by cleavage of one or more peptide bonds, which then serves as the activating protease for the next zymogen molecule in the cascade. In the final proteolytic step of the cascade, fibrinogen is proteolytically cleaved by thrombin to fibrin, which is then crosslinked at the site of injury to form a clot.

As used herein, “hemostasis” may refer to the stopping or slowing of bleeding or blood flow in an organ or body part. The term hemostasis can encompass the entire process of blood clotting to prevent or reduce blood loss following blood vessel injury to subsequent dissolution of the blood clot following tissue repair.

As used herein, “clotting” or “coagulation” refers to the formation of an insoluble fibrin clot, or the process by which the coagulation factors of the blood interact in the coagulation cascade, ultimately resulting in the formation of an insoluble fibrin clot.

As used herein, human tissue factor comprises the amino acid sequence as set forth in GenBank Accession No. NP_{—}001984.1 or NP_{—}001171567.1 (SEQ ID NO: 2) as well as proteins encoded by the sequences set forth in GenBank Accession No. NM_{—}001993.4 (SEQ ID NO: 3) and NM_{—}001178096.1 (SEQ ID NO: 4). When modification positions are described herein, the positions are in reference to GenBank Accession No. NP_{—}001984.1 (SEQ ID NO: 1).

As used herein an “analogue” of tissue factor is a compound which retains the sequence of tissue factor necessary for the functional activity of tissue factor, yet which also contains certain modifications. For definitional purposes, the functional activity of tissue factor is the initiation of clotting within 15 seconds in a prothrombin time assay. An example of an analogue of tissue factor is a protein which retains the functional activity of tissue factor but includes one or more non-naturally-occurring amino acids and in one example 1 substituted amino acid, 2 substituted amino acids, 5 substituted amino acids, up to 10 substituted amino acids, up to 20 substituted amino acids or up to 30 substituted amino acids. In certain embodiments, these modifications are within the extracellular domain of tissue factor. Modifications can be particularly located between positions 33 and 251 or more particularly between positions 45-155. The term “analogue” is also intended to include modified allelic variants of tissue factor.

As used herein, a “homologue” refers to tissue factor from a species other than human wherein the sequence of the non-human tissue factor has at least 85% sequence identity with human tissue factor, at least 90% sequence identity with human tissue factor, at least 95% sequence identity with human tissue factor or at least 99% sequence identity with human tissue factor.

As used herein a “precursor” of tissue factor is a molecule that can be cleaved to form tissue factor, or an analogue, homologue or derivative thereof and/or that can be altered from an inactive to an active state.

As used herein, a “derivative” of tissue factor is a portion of the tissue factor protein, a portion of a tissue factor analogue, a portion of a tissue factor homologue or a portion of a tissue factor precursor. Derivatives include fragments. In particular embodiments, the derivative is a 263 amino acid fragment of SEQ ID NO: 1 or 2. The 263 amino acid fragment of SEQ ID NO; 1 or 2 can begin at: position 1 and end at position 263; position 2 and end at position 264; position 3 and end at position 265; position 4 and end at position 266; position 5 and end at position 267; position 6 and end at position 268; position 7 and end at position 269; position 8 and end at position 270; position 9 and end at position 271; position 10 and end at position 272; position 11 and end at position 273; position 12 and end at position 274; position 13 and end at position 275; position 14 and end at position 276; position 15 and end at position 277; position 16 and end at position 278; position 17 and end at position 279; position 18 and end at position 280; position 19 and end at position 281; position 20 and end at position 282; position 21 and end at position 283; position 22 and end at position 284; position 23 and end at position 285; position 24 and end at position 286; position 25 and end at position 287; position 26 and end at position 288; position 27 and end at position 289; position 28 and end at position 290; position 29 and end at position 291; position 30 and end at position 292; position 31 and end at position 293; position 32 and end at position 294; or position 33 and end at position 295. A derivative also includes fragments including amino acids 33-251 and 45-155.

Tissue factor (factor III) is one of the most rapid initiators of blood coagulation is a cell-surface protein, tissue factor. Tissue factor is a transmembrane protein that contains an extracellular domain, and is expressed on most cells outside of the vasculature. In a particular embodiment, a tissue factor precursor is 295 amino acids. Without being bound by theory or mechanism of action, exposure of tissue factor to blood initiates the extrinsic coagulation pathway by binding to a serine protease, factor VIIa (see FIG. 1). Although tissue factor itself is not a protease, binding of factor VIIa to the extracellular domain of tissue factor increases the rate of cleavage of both factor X and factor IX by factor VIIa. Whereas kaolin-induced coagulation through the intrinsic pathway typically takes ˜28-44 seconds in an activated partial thromboplastin assay, tissue factor-induced coagulation through the extrinsic pathway takes only ˜12-15 seconds in a prothrombin time assay. Soluble recombinant forms of the extracellular domain of tissue factor are now marketed for use in PT assays. Recombinant tissue factor containing 219 amino acids of the extracellular domain and from between 100 and 110 amino acids of the extracellular domain is commercially available.

Depending upon the nature of the wound and the treatment method employed, one or more hemostatic agents (e.g., TF) optionally with one or more phospholipids can be coupled with a hemostatic material and fabricated in various forms. For example, a puff, fleece, gel, powder, or sponge form can be used for controlling active bleeding from an artery or vein, or for controlling internal bleeding during laparoscopic procedures. In neurosurgery, where oozing brain wounds are commonly encountered, a sheet or towel form of the hemostatic material can be used. Likewise, in oncological surgery, especially of the liver, it can be useful to employ a sheet form or sponge form of the hemostatic material, which can be placed in or on the tumor bed to control oozing. In dermatological applications, a sheet form can be used. In closing punctures in a blood vessel, a puff or fleece form can generally be used. A suture form, especially a microsuture form, can be used in certain applications. Despite differences in delivery and handling characteristics of the various forms, the wound dressings disclosed herein can each be effective in deploying one or more hemostatic agents (e.g., TF) optionally with one or more phospholipid co-factors to an affected site and rapidly initiating hemostatic plug formation through platelet adhesion, platelet activation, and/or blood coagulation.

The hemostatic material used in wound dressings disclosed herein can be formed from any appropriate material. For instance, some non-limiting examples include cellulose, cellulose derivatives (e.g. alkyl cellulose (e.g., methyl cellulose), hydroxyalkyl cellulose, alkylhydroxyalkyl cellulose, cellulose sulfate, salts of carboxymethyl cellulose, carboxymethyl cellulose, and carboxyethyl cellulose), chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, heparin, heparin sulfate, heparan, heparan sulfate, dermatan sulfate, keratin sulfate, carrageenans, chitosan, starch, amylose, amylopectin, poly-N-glucosamine, polymannuronic acid, polyglucuronic acid, polyguluronic acid and derivatives of the above.

Employed hemostatic materials of particular embodiments generally exhibit good adherence to wounds such that an adhesive, for example, a pressure sensitive adhesive, is generally not necessary. However, for ease of use and to ensure that the hemostatic material remains in a fixed position after application to the wound, pressure sensitive adhesives can be used. For example, to ensure that the hemostatic material remains affixed to the wound, a suitable adhesive can be employed, for example, along the edges or a side of the hemostatic fabric, sponge or puff. Although any adhesive suitable for forming a bond with skin or other tissue can be used, in certain embodiments a pressure sensitive adhesive is used. Pressure sensitive adhesives are generally defined as adhesives that adhere to a substrate when a light pressure is applied but leave little to no residue when removed. Pressure sensitive adhesives include, but are not limited to, solvent in solution adhesives, hot melt adhesives, aqueous emulsion adhesives, calenderable adhesives, and radiation curable adhesives. Hot melt adhesives can typically be based on resin-tackified block copolymers. Aqueous emulsion adhesives can include those prepared using acrylic copolymers, butadiene styrene copolymers, and natural rubber latex. Radiation curable adhesives can typically consist of acrylic oligomers and monomers, which can cure to form a pressure sensitive adhesive upon exposure to ultraviolet lights.

The most commonly used elastomers in pressure sensitive adhesives can include natural rubbers, styrene-butadiene latexes, polyisobutylene, butyl rubbers, acrylics, and silicones. In particular embodiments, acrylic polymer or silicone-based pressure sensitive adhesives can be used. Acrylic polymers can often have a low level of allergenicity, be cleanly removable from skin, possess a low odor, and exhibit low rates of mechanical and chemical irritation. Medical grade silicone pressure sensitive adhesives can be chosen for their biocompatibility.

Amongst the factors that influence the suitability for a pressure sensitive adhesive for use in wound dressings of particular embodiments is the absence of skin irritating components, sufficient cohesive strength such that the adhesive can be cleanly removed from the skin, ability to accommodate skin movement without excessive mechanical skin irritation, and good resistance to body fluids.

In particular embodiments, the pressure sensitive adhesive can comprise a butyl acrylate. While butyl acrylate pressure sensitive adhesives can generally be used for many applications, any pressure sensitive adhesive suitable for bonding skin can be used. Such pressure sensitive adhesives are well known in the art.

While the hemostatic puffs, fabrics and other hemostatic materials generally exhibit good mechanical strength and wound protection, in certain embodiments, it can be useful to employ a backing or other material on one side of the hemostatic material. For example, a composite including two or more layers can be prepared, wherein one of the layers is the hemostatic material and another layer an elastomeric layer, gauze, vapor-permeable film, waterproof film, a woven or nonwoven fabric, a mesh, or the like. The layers can then be bonded using any suitable method (e.g., the application of adhesives, such as pressure sensitive adhesives, hot melt adhesives, curable adhesives; the application of heat or pressure, such as in lamination, a physical attachment through the use of stitching, studs, other fasteners; or the like).

Other components can be combined with the hemostatic materials for use in wound dressings as are known in the art, such as preservatives, stabilizers, dyes, buffers, alginate pastes or beads, hydrocolloid pastes or beads, hydrogel pastes or beads, as well as medicaments and other therapeutic agent as described below.

In other embodiments, the hemostatic material can be mixed with any suitable liquid to produce a hemostatic gel. The gel can be used inside and outside the body.

Hemostatic agents including, for example, clotting factors (e.g., tissue factor and/or analogues, homologues, precursors or derivatives thereof as well as all hemostatic agents described above) used with wound dressings disclosed herein can be derived from any available source including plasma-derived, recombinantly-produced and/or that which is commercially available. For example, the soluble recombinant forms of the extracellular domain of tissue factor are available under the tradename Innovin® by Dade Behring, Deerfield, Ill. and ProSpec (219 amino acids of the extracellular domain (33-251) with a molecular weight of 29.39 kDa).

A phospholipid for use in the wound dressings described herein includes synthetic, semi-synthetic and naturally-occurring phospholipids and derivatives thereof. Phospholipids appropriate for use with embodiments of the present disclosure include those of, without limitation, human, animal or vegetable origin. Non-limiting exemplary phospholipids include phosphatidylethanolamine, phosphatidylcholine, phosphatidylserine, phosphatidylinositol, phosphatidylglycerin, cardiolipin, sphingomyelin, ceramide phosphorylethanolamine, ceramide phosphorylglycerin, ceramide phosphorylglycerin phosphate, 1,2-dimyristoyl-1,2-deoxyphosphatidylcholine, plasmalogen, phosphatidic acid and the like, and these phospholipids may be used alone or in various combinations.

on-limiting exemplary phospholipid derivatives include 2-Didecanoyl-sn-glycero-3-phosphocholine (DDPC); 1,2-Dierucoyl-sn-glycero-3-phosphate (Sodium Salt) (DEPA-NA); 1,2-Dierucoyl-sn-glycero-3-phosphocholine (DEPC); 1,2-Dierucoyl-sn-glycero-3-phosphoethanolamine (DEPE); 1,2-Dierucoyl-sn-glycero-3[Phospho-rac-(1-glycerol) (Sodium Salt) (DEPG-NA); 1,2-Dilinoleoyl-sn-glycero-3-phosphocholine (DLOPC); 1,2-Dilinoleoyl-sn-glycero-3-phosphocholine (DLOPC); 1,2-Dilauroyl-sn-glycero-3-phosphate (Sodium Salt) (DLPA-NA); 1,2-Dilauroyl-sn-glycero-3-phosphocholine (DLPC); 1,2-Dilauroyl-sn-glycero-3-phosphoethanolamine (DLPE); 1,2-Dilauroyl-sn-glycero-3[Phospho-rac-(1-glycerol) (Sodium Salt) (DLPG-NA); 1,2-Dilauroyl-sn-glycero-3[Phospho-rac-(1-glycerol) (Ammonium Salt) (DLPG-NH4); 1,2-Dilauroyl-sn-glycero-3-phosphoserine (Sodium Salt) (DLPS-NA); 1,2-Dimyristoyl-sn-glycero-3-phosphate (Sodium Salt) (DMPA-NA); 1,2-Dimyristoyl-sn-glycero-3-phosphocholine (DMPC); 1,2-Dimyristoyl-sn-glycero-3-phosphoethanolamine (DMPE); 1,2-Dimyristoyl-sn-glycero-3[Phospho-rac-(1-glycerol) (Sodium Salt) (DMPG-NA); 1,2-Dimyristoyl-sn-glycero-3[Phospho-rac-(1-glycerol . . . ) (Ammonium Salt) (DMPG-NH4); 1,2-Dimyristoyl-sn-glycero-3[Phospho-rac-(1-glycerol) (Sodium/Ammonium Salt) (DMPG-NH4/NA); 1,2-Dimyristoyl-sn-glycero-3-phosphoserine (Sodium Salt) (DMPS-NA); 1,2-Dioleoyl-sn-glycero-3-phosphate (Sodium Salt) (DOPA-NA); 1,2-Dioleoyl-sn-glycero-3-phosphocholine (DOPC); 1,2-Dioleoyl-sn-glycero-3-phosphoethanolamine (DOPE); 1,2-Dioleoyl-sn-glycero-3[Phospho-rac-(1-glycerol) (Sodium Salt) (DOPG-NA); 1,2-Dioleoyl-sn-glycero-3-phosphoserine (Sodium Salt) (DOPS-NA); 1,2-Dipalmitoyl-sn-glycero-3-phosphate (Sodium Salt) (DPPA-NA); 1,2-Dipalmitoyl-sn-glycero-3-phosphocholine (DPPC); 1,2-Dipalmitoyl-sn-glycero-3-phosphoethanolamine (DPPE); 1,2-Dipalmitoyl-sn-glycero-3[Phospho-rac-(1-glycerol) (Sodium Salt) (DPPG-NA); 1,2-Dipalmitoyl-sn-glycero-3[Phospho-rac-(1-glycerol) (Ammonium Salt) (DPPG-NH4); 1,2-Dipalmitoyl-sn-glycero-3-phosphoserine (Sodium Salt) (DPPS-NA); 1,2-Distearoyl-sn-glycero-3-phosphate (Sodium Salt) (DSPA-NA); 1,2-Distearoyl-sn-glycero-3-phosphocholine (DSPC); 1,2-Distearoyl-sn-glycero-3-phosphoethanolamine (DSPE); 1,2-Distearoyl-sn-glycero-3[Phospho-rac-(1-glycerol) (Sodium Salt) (DSPG-NA); 1,2-Distearoyl-sn-glycero-3[Phospho-rac-(1-glycerol) (Ammonium Salt) (DSPG-NH4); 1,2-Distearoyl-sn-glycero-3-phosphoserine (Sodium Salt) (DSPS-NA); Egg-PC (EPC); Hydrogenated Egg PC (HEPC); High purity Hydrogenated Soy PC (HSPC); Hydrogenated Soy PC (HSPC); 1-Myristoyl-sn-glycero-3-phosphocholine (LYSOPC MYRISTIC); 1-Palmitoyl-sn-glycero-3-phosphocholine (LYSOPC PALMITIC); 1-Stearoyl-sn-glycero-3-phosphocholine (LYSOPC STEARIC); 1-Myristoyl-2-palmitoyl-sn-glycero 3-phosphocholine (Milk Sphingomyelin MPPC); 1-Myristoyl-2-stearoyl-sn-glycero-3-phosphocholine (PMPC); 1-Palmitoyl-2-oleoyl-sn-glycero-3-phosphocholine (POPC); 1-Palmitoyl-2-oleoyl-sn-glycero-3-phosphoethanolamine (POPE); 1-Palmitoyl-2-oleoyl-sn-glycero-3[Phospho-rac-(1 -glycerol)] (Sodium Salt) (POPG-NA); -Palmitoyl-2-stearoyl-sn-glycero-3-phosphocholine (PSPC); 1-Stearoyl-2-myristoyl-sn-glycero-3-phosphocholine (SMPC); 1-Stearoyl-2-oleoyl-sn-glycero-3-phosphocholine (SOPC); and 1-Stearoyl-2-palmitoyl-sn-glycero-3-phosphocholine (SPPC).

For recombinant expression of one or more hemostatic proteins (e.g., TF), the nucleic acid containing all or a portion of the nucleotide sequence encoding the hemostatic protein including, for example, a cDNA coding for TF (e.g., GenBank Accession No. NM_{—}001993.4 or NM_{—}001178096.1) can be inserted into an appropriate expression vector, i.e., a vector that contains the necessary elements for the transcription and translation of the inserted protein coding sequence. Exemplary of such a vector is any mammalian expression vector such as, for example, pCMV. The necessary transcriptional and translational signals also can be supplied by the native promoter for the gene coding for the hemostatic protein, and/or their flanking regions.

Also provided are vectors that contain nucleic acid encoding a hemostatic protein (e.g., TF or modified TF). Cells containing the vectors also are provided. The cells include eukaryotic and prokaryotic cells. Such cells include bacterial cells, yeast cells, fungal cells, Archea, plant cells, insect cells and animal cells. The cells are used to produce a hemostatic polypeptide or modified hemostatic polypeptide thereof by growing the above-described cells under conditions whereby the encoded hemostatic protein is expressed by the cell, and recovering the expressed hemostatic protein. For purposes herein, the hemostatic can be secreted into the medium.

Modified hemostatic polypeptides such as modified TF can be incorporated in the wound dressings disclosed herein. These hemostatic polypeptides exhibit alterations in one or more activities or properties compared with an unmodified hemostatic polypeptide. The activities or properties that can be altered as a result of modification include, but are not limited to, coagulation or coagulant activity; pro-coagulant activity; proteolytic or catalytic activity; antigenicity; half-life; three-dimensional structure; pl; and/or conformation. Typically, the modified hemostatic polypeptides exhibit procoagulant activity. Such modified hemostatic polypeptides can be used in the treatment of unwanted or uncontrolled bleeding where hemostatic polypeptides can function to promote blood coagulation.

Modifications in a hemostatic polypeptide can be made to any form of a hemostatic polypeptide, including allelic and species variants, splice variants, variants known in the art, or hybrid or chimeric hemostatic molecules. For example, the modifications provided herein can be made in a precursor hemostatic polypeptide.

Modifications provided herein of a starting, unmodified reference polypeptide include amino acid replacements or substitution, additions or deletions of amino acids, or any combination thereof. For example, modified hemostatic polypeptides include those with 1, 2, 3, 4, 5, 6, 7, 8, 9, 10, 11, 12, 13, 14, 15, 16, 17, 18, 19, 20, 30, 40, 50 or more modified positions. Also provided herein are modified hemostatic polypeptides with two or more modifications compared to a starting reference hemostatic polypeptide. Any modification provided herein can be combined with any other modification known to one of skill in the art so long as the resulting modified hemostatic polypeptide exhibits coagulation activity.

The modifications provided herein can be made by standard recombinant DNA techniques such as are routine to one of skill in the art. Any method known in the art to effect mutation of any one or more amino acids in a target protein can be employed. Methods include standard site-directed mutagenesis (using e.g., a kit, such as kit such as QuikChange® available from Agilent Technologies, Inc., Santa Clara, Calif.) of encoding nucleic acid molecules, or by solid phase polypeptide synthesis methods. In addition, modified chimeric proteins (i.e. Gla domain swap) can be generated by routine recombinant DNA techniques. For example, chimeric polypeptides can be generated using restriction enzymes and cloning methodologies for routine subcloning of the desired chimeric polypeptide components.

Other modifications that are or are not in the primary sequence of the polypeptide also can be included in a modified hemostatic polypeptide, or conjugate thereof, including, but not limited to, the addition of a carbohydrate moiety, the addition of a polyethylene glycol (PEG) moiety, the addition of an Fc domain, etc. For example, such additional modifications can be made to increase the stability or half-life of the protein.

Fusion proteins containing a modified or unmodified hemostatic polypeptide and one or more other polypeptides also are provided. Linkage of a hemostatic polypeptide with another polypeptide can be affected directly or indirectly via a linker. In one example, linkage can be by chemical linkage, such as via heterobifunctional agents or thiol linkages or other such linkages. Fusion also can be affected by recombinant means. Fusion of a hemostatic polypeptide to another polypeptide can be to the N- or C-terminus of the hemostatic polypeptide.

A fusion protein can be produced by standard recombinant techniques. For example, DNA fragments coding for the different polypeptide sequences can be ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence. Moreover, many expression vectors are commercially available that already encode a fusion moiety (e.g., a GST polypeptide). A hemostatic polypeptide-encoding nucleic acid can be cloned into such an expression vector such that the fusion moiety is linked in-frame to the protease protein.

All dressings disclosed herein incorporate one or more hemostatic agents (e.g., tissue factor and/or analogues, homologues, precursors or derivatives thereof) and optionally one or more phospholipids. Dressings can also incorporate one or more other active components that additionally promote clotting and/or serve a different purpose. For example, additional active components that promote clotting include, without limitation, other plasma purified or recombinant coagulation factors, procoagulants, such as vitamin K, vitamin K derivative and protein C inhibitors, plasma, platelets, red blood cells, corticosteroids, kaolin, chitosan, zeolite, fibrin, smectite, minerals, clotting factor concentrates, recombinant Factor VIIa, alphanate FVIII concentrate, bioclate FVIII concentrate, monoclate-P FVIII concentrate, haemate P FVIII, von Willebrand factor concentrate, helixate FVIII concentrate, hemophil-M FVIII concentrate, humate-P FVIII concentrate, porcine FVIII concentrate, koate HP FVIII concentrate, kogenate FVIII concentrate, recombinate FVIII concentrate, mononine FVIIa concentrate, and fibrogammin P FXIII concentrate. To the extent not expressly included in the definition above, each of these components should also be considered a hemostatic agent.

In addition to effectively delivering a hemostatic agent to a wound, in some embodiments, the hemostatic materials can deliver other substances as well. In a particularly useful embodiment, such substances can include medicaments, pharmaceutical compositions, therapeutic agents, and/or other substances producing a physiological effect. Some non-limiting examples of suitable anti-inflammatory agents include, but are not limited to, nonsteroidal anti-inflammatory drugs (NSAIDs), such as aspirin, celecoxib, choline magnesium trisalicylate, diclofenac potasium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids, such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

Anti-infective agents can include, but are not limited to, anthelmintics (mebendazole), antibiotics including aminoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim.

Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocalne, and phenazopyridine.

Cell proliferative agents, such as tretinoin; procoagulants, such as dencichine (2-amino-3-(oxalylamino)-propionic acid); and sunscreens, such as oxybenzone and octocrylene can also be used.

Human epidermal growth factor (hEGF) can also be useful for certain embodiments. This small molecular weight peptide is a mitogenic protein and can be critical for skin and epidermal regeneration. It can be a small 53 amino acid residue long protein with 3 disulfide bridges. The epidermal growth factor can be used as produced, or can be polymerized prior to use in particular embodiments. Presence of hEGF can have a positive effect upon skin healing and regeneration.

Other substances which can be used in particular embodiments can include, or be derived from, traditional medicaments, agents, and remedies that have known antiseptic, wound healing, and pain relieving properties. These agents can include, but are not limited to, Sanqi (Radix Notoginsent). Another such agent can be Dahuang (Radix Et Rhizoma Rhei). One of its compounds, Emodin, can cause anti-inflammatory effects and can also effectively reduce soft tissue edema. Another agent can include Zihuaddng (Herba Violae), which has been used as an antibiotic agent.

Baiji (Rhizoma Bletillae) has been used as a hemostatic agent and also to promote wound. It can contain the following substances: (3,3′-di-hydroxy-2′,6′-bis(p-hydroxybenzyl)-5-methoxybibenzy-1); 2,6-bis(p-hydroxybenzyl)-3′,5-dimethoxy-3-hydroxy-bibenzyl); (3,3′-dihydroxy-5-methoxy-2,5′,6-tris(p-hydroxy-benzyl)bibenzyl; 7-dihydroxy-1-p-hydroxybenzyl-2-methoxy-9,10-dihydro-phenanthrene); (4,7-dihydroxy-2-methoxy-9,10-dihydroxyphenanthrene); Blestriarene A (4,4′-dimethoxy-9,9′,10,10′-tetrahydro[1,1′-biphenanthrene]-2,2′,7,7′-te-trol); Blestriarene B (4,4′-dimethoxy-9,10-dihydro[1,1′-biphenanthrene]-2-,2′,7,7′-tetrol); Batatasin; 3′-O-Methyl Batatasin; Blestrin A(1); Blestrin B(2); Blestrianol A (4,4′-dimethoxy-9,9′,10,10′-tetrahydro]-1′,3-1-biphenanthrene]-2,2′,7,7′-tetraol); Blestranol B (4′,5-dimethoxy-8-(4-hyd-roxybenzyl)-9,9′,10,10′-tetrahydro-[1,3-biphenanthrene]-2,2′,7,7′-tetraol-Blestranol C (4′,5′-dimethoxy-8-(4-hydroxybenzyl)-9,10-dihydro-[1′,3-bi-phenanthrene]-2,2′,7,7′-tetraol); (1,8-bi(4-hydroxybenzyl)-4-methoxy-phena-nthrene-2,7-diol); 3-(4-hydroxybenzyl)-4-methoxy-9,10-dihydro-phenanthrene-2,7-diol; (1,6-bi(4-hydroxybenzyI)-4-methoxy-9,10-dihydro-phenanthrene-2,-7-diol; (1-p-hydroxybenzyl-4-methoxyphenanthrene-2,7-diol); 2,4,7-trimethoxy-phenanthrene; 2,4,7-trimethoxy-9,10-dihydrophenanthrene; 2,3,4,7-tetramethoxyphenanthrene; 3,3′,5-trimethoxy-bibenzyl; 3,5-dimethoxybibenzyl; and Physcion.

Rougui (Cortex Cinnamoni) has pain relief effects. It can contain some or all of the following substances: anhydrocinnzeylanine; anhydrocinnzeylanol; cinncassiol A; cinnacassiol A monoacetate; cinncassiol A glucoside; cinnzeylanine; cinnzeylanol; cinncassiol B glucoside; cinncassiol C₁; cinncassiol C₁ glucoside; cinncassiol C₂; cinncassiol C₂; cinncassiol D₁; cinncassiol D₁ glucoside; cinncassiol D₂; cinncassiol D₂ glucoside; cinncassiol D₃; cinncassiol D₄; cinncassiol D₄ glucoside; cinncassiol E; lyoniresinol; 3α-O-B-D-glucopyranoside; 3,4,5-trimethoxyphenyl 1-O-β-D-apiofuranosyl-(1/6)-β-D-glucopyranoside; (±)-syringaresinol; cinnamic aldehyde cyclic glycerol 1,3 acetals; epicatechin; 3′-O-methyl-(−)-epicatechin; 5,3′-di-O-methyl-(−)-epicatechin-; 5,7,3′-tri-O-methyl-(−)-epicatechin, 5′-O-methyl-(+)-catechin; 7,4′-di-O-methyl-(+)-catech in; 5,7,4′-tri-O-methyl-(+)-catechin; (−)-epicatechin-3-O-β-D-glucopyranoside; (−)-epicatechin-8-C-β-D-glucopyranoside; (−)-epicatechin-6-C-β-D-glucopyranoside; procyanidin; cinnamtannin A₂, A₃, A₄; (−)-epicatechin; procyanidins B-1, B-2, B-5, B-7, C-1; proanthocyanidin; proanthocyanidin A-2; 8-C-β-D-glucopyranoside; procyanidin B-2 8-C-β-D-glycopyranoside; cassioside[(4s)-2,4-dimethyl-3-(4-hydroxy-3-hydroxymethyl-1-butenyl)-4-(β-D-glucopyranosyl)methyl-2-cyclohexen-1-one]; 3,4,5-trimethoxyphenyl-β-D-apiofuranosyl-[(1.f-wdarw.6)-β-D-glucopyranoside; coumarin; cinnamic acid; procyanidin; procyanidin B₂; cinnamoside[(3R)-4-{(2′R,4′S)-2′-hydroxy-4′-(β-1-D-apiofuranoxy-(1/6)-β-D-glucopyranosyl)-2′,6′,6′-trimethyl-cyclohexylidene}-3-buten-2-one]; cinnamaldehyde; 3-2(hydroxyphenyl)-propano-ic acid; 0-glucoside; cinnaman A₂; P, S, Cl, K, Ca, Ti, Mn, Fe, Cu, Zn, Br, Rb, Sr, and Ba.

Other substances that can be incorporated into the hemostatic material of particular embodiments can include various pharmacological agents, excipients, and other substances well known in the art of pharmaceutical formulations. Other pharmacological agents can include, but are not limited to, ACE inhibitors, and cytotoxic agents. These other substances can include ionic and nonionic surfactants (e.g., Pluronic®, BASF Corporation, Mount Olive, N.J.; Triton™, Dow Chemical Company, Midland, Mich.), detergents (e.g., polyoxyl stearate, sodium lauryl sulfate), emulsifiers, demulsifiers, stabilizers, aqueous and oleaginous carriers (e.g., white petrolatum, isopropyl myristate, lanolin, lanolin alcohols, mineral oil, sorbitan monooleate, propylene glycol, cetylstearyl alcohol), emollients, solvents, preservatives (e.g., methylparaben, propylparaben, benzyl alcohol, ethylene diamine tetraacetate salts), thickeners (e.g., pullulin, xanthan, polyvinylpyrrolidone, carboxymethylcellulose), plasticizers (e.g., glycerol, polyethylene glycol), antioxidants (e.g., vitamin E, vitamin K, vitamin C, calcium), buffering agents, flexible agents (e.g., silicon), antibiotics, low-grade antibiotics (e.g., silver, tetracycline, etc.), and the like.

The dressings disclosed herein can be presented in a package, in a kit or other device that can contain one or more unit dosage forms. The dressings can be accompanied by instructions for application.

Denaturation of impregnated hemostatic agent (e.g., tissue factor) can be reduced or avoided in particular embodiments by flash freezing and lyophilization, techniques well-known to those of ordinary skill in the art. Particular embodiments can utilize dressings that are hermetically sealed in anaerobic packaging for field use.

The following summarizes non-limiting exemplary embodiments of wound dressings, methods of using the wound dressings and associated kits described herein:

Exemplary Embodiment 1. A fast-clotting wound dressing impregnated with tissue factor and/or analogues, homologues, precursors or derivatives thereof.

Exemplary Embodiment 2. The fast-clotting wound dressing of embodiment 1, wherein the tissue factor is human tissue factor.

Exemplary Embodiment 3. The fast-clotting wound dressing of embodiment 2, wherein the human tissue factor is produced using recombinant technology.

Exemplary Embodiment 4. The fast-clotting wound dressing of embodiment 1, 2 or 3, wherein the wound dressing initiates clotting within 120, 100, 80, 60, 50, 40, 35, 30, 27, 26, 25, 24, 23, 22, 21, 20, 19, 18, 17, 16, 15, 14, 13, 12, 11, 10, 9, 8, 7, 6, 5, 4, 3, 2, or 1 second(s) of application to a wound.

Exemplary Embodiment 5. The fast-clotting wound dressing of embodiment 1, 2 or 3, wherein the wound dressing initiates clotting within 27 seconds of application to a wound.

Exemplary Embodiment 6. The fast-clotting wound dressing of embodiment 1, 2, 3, 4 or 5 wherein tissue factor is impregnated at a concentration of 5 pmoles of recombinant tissue factor per bandage, 10 pmoles (0.34 μg) of recombinant tissue factor per bandage, 20 pmoles per bandage, 30 pmoles per bandage, 40 pmoles per bandage, 50 pmoles per bandage, 60 pmoles per bandage, 70 pmoles per bandage, 80 pmoles per bandage, 90 pmoles per bandage, or 100 pmoles per bandage.

Exemplary Embodiment 7. The fast-clotting wound dressing of embodiment 1, 2, 3, 4, 5 or 6, wherein the dressing is further impregnated with a secondary clotting factor.

Exemplary Embodiment 8. The fast-clotting wound dressing of embodiment 1, 2, 3, 4, 5, 6, or 7 wherein the dressing is further impregnated with pharmaceuticals, antifungal agents, topical pain reducing medication, pharmaceuticals, anti-inflammatory agents and/or tissue enzyme inhibitors.

Exemplary Embodiment 9. The fast-clotting wound dressing of embodiment 8, wherein the pharmaceutical is an analgesic, an anti-inflammatory agent, or an antibacterial.

Exemplary Embodiment 10. A method for treating an individual having a wound where blood is present comprising applying the fast-clotting wound dressing of any one of embodiments 1 to 9 to the wound on the individual.

Exemplary Embodiment 11. The method of embodiment 10, wherein the individual has an acquired coagulopathy.

Exemplary Embodiment 12. The method of embodiment 10 or 11, wherein the individual has a traumatic injury which causes bleeding.

Exemplary Embodiment 13. The method of embodiment 10, 11 or 12 wherein the individual is undergoing a surgical procedure.

Exemplary Embodiment 14. A method to initiate blood clotting on a wound of a subject in need thereof comprising applying a fast-clotting wound dressings impregnated with tissue factor and/or analogues, homologues, precursors or derivatives thereof to the wound.

Exemplary Embodiment 15. The method of embodiment 14, wherein the tissue factor is human tissue factor.

Exemplary Embodiment 16. The method of embodiment 15, wherein the human tissue factor is recombinantly produced.

Exemplary Embodiment 17. The method of embodiment 14, 15 or 16, wherein the wound dressing initiates clotting within 120, 100, 80, 60, 50, 40, 35, 30, 27, 26, 25, 24, 23, 22, 21, 20, 19, 18, 17, 16, 15, 14, 13, 12, 11, 10, 9, 8, 7, 6, 5, 4, 3, 2, or 1 second(s) of application to a wound.

Exemplary Embodiment 18. The method of embodiment 14 15 or 16, wherein the wound dressing initiates clotting within 27 seconds of application to a wound.

Exemplary Embodiment 19. The method of embodiment 14, 15, 16, 17 or 18 wherein tissue factor is impregnated at a concentration of 5 pmoles of recombinant tissue factor per bandage, 10 pmoles (0.34 μg) of recombinant tissue factor per bandage, 20 pmoles per bandage, 30 pmoles per bandage, 40 pmoles per bandage, 50 pmoles per bandage, 60 pmoles per bandage, 70 pmoles per bandage, 80 pmoles per bandage, 90 pmoles per bandage, or 100 pmoles per bandage.

Exemplary Embodiment 20. The method of embodiment 14, 15, 16, 17, 18 or 19 wherein the dressing is further impregnated with a secondary clotting factor.

Exemplary Embodiment 21.The method of embodiment 14, 15, 16, 17, 18, 19 or 20, wherein the dressing is further impregnated with pharmaceuticals, antifungal agents, topical pain reducing medication, pharmaceuticals, anti-inflammatory agents and/or tissue enzyme inhibitors.

Exemplary Embodiment 22. The method of embodiment 21, wherein the pharmaceutical is an analgesic, an anti-inflammatory agent, or an antibacterial.

Exemplary Embodiment 23. The method of embodiment 14, wherein the wound comprises broken skin tissue.

Exemplary Embodiment 24. The method of embodiment 14, wherein the wound comprises broken soft tissue within a human body.

Exemplary Embodiment 25. A kit for initiating blood clotting, the kit comprising a fast-clotting wound dressing impregnated with tissue factor and/or analogues, homologues, precursors or derivatives thereof; and instructions for using the kit.

Exemplary Embodiment 26. A fast-clotting wound dressing impregnated with tissue factor and/or analogues, homologues, precursors or derivatives thereof wherein the wound dressing initiates clotting within 1 second-2 minutes of application to a wound or within any time in between 1 second-2 minutes.

Exemplary Embodiment 27. A fast-clotting wound dressing impregnated with one or more hemostatic agents and one or more phospholipids or derivatives thereof.

Exemplary Embodiment 28. The fast-clotting wound dressing of embodiment 27, wherein the hemostatic agent is tissue factor and/or analogues, homologues, precursors or derivatives thereof.

Exemplary Embodiment 29. The fast-clotting wound dressing of embodiment 28, wherein the tissue factor is human tissue factor.

Exemplary Embodiment 30. The fast-clotting wound dressing of embodiment 29, wherein the human tissue factor is produced using recombinant technology.

Exemplary Embodiment 31. The fast-clotting wound dressing of embodiment 27, wherein said wound dressing initiates clotting within 120 seconds of application to a wound.

Exemplary Embodiment 32. The fast-clotting wound dressing of embodiment 27, wherein said wound dressing initiates clotting within 27 seconds of application to a wound.

Exemplary Embodiment 33. The fast-clotting wound dressing of embodiment 27, wherein the hemostatic agent is impregnated at a concentration of 5 pmoles-100 pmoles per bandage.

Exemplary Embodiment 34. The fast-clotting wound dressing of embodiment 27, wherein the dressing is further impregnated with an additional clotting factor.

Exemplary Embodiment 35. The fast-clotting wound dressing of embodiment 27, wherein the dressing is further impregnated with one or more agents selected from the group consisting of pharmaceuticals, antifungal agents, topical pain reducing medication, pharmaceuticals, anti-inflammatory agents and tissue enzyme inhibitors.

Exemplary Embodiment 36. The fast-clotting wound dressing of embodiment 35, wherein the pharmaceutical is an analgesic, an anti-inflammatory agent, or an antibacterial.

Exemplary Embodiment 37. A method for treating an individual having a wound where bleeding is present comprising applying the fast-clotting wound dressing of any one of embodiments 27 to 36, 57 or 58 to the wound on the individual.

Exemplary Embodiment 38. The method of embodiment 37, wherein the individual has an acquired coagulopathy.

Exemplary Embodiment 39. The method of embodiment 37, wherein the individual has a traumatic injury which causes the bleeding.

Exemplary Embodiment 40. The method of embodiment 37, wherein the individual is undergoing a surgical procedure.

Exemplary Embodiment 41. A method to initiate blood clotting on a wound of a subject in need thereof comprising applying a fast-clotting wound dressing impregnated with a hemostatic agent and a phospholipid to the wound.

Exemplary Embodiment 42. The method of embodiment 41, wherein the hemostatic agent is tissue factor.

Exemplary Embodiment 43. The method of embodiment 42, wherein the tissue factor is human tissue factor.

Exemplary Embodiment 44. The method of embodiment 43, wherein the human tissue factor is recombinantly produced.

Exemplary Embodiment 45. The method of embodiment 41, wherein said wound dressing initiates clotting within 1-27 second(s) of application to a wound.

Exemplary Embodiment 46. The method of embodiment 41, wherein said wound dressing initiates clotting within 27 seconds of application to a wound.

Exemplary Embodiment 47. The method of embodiment 41, wherein tissue factor is impregnated at a concentration of 5 pmoles-100 pmoles per bandage.

Exemplary Embodiment 48. The method of embodiment 41, wherein the dressing is further impregnated with an additional clotting factor.

Exemplary Embodiment 49. The method of embodiment 41, wherein the dressing is further impregnated with one or more agents selected from the group consisting of pharmaceuticals, antifungal agents, topical pain reducing medication, pharmaceuticals, anti-inflammatory agents and/or tissue enzyme inhibitors.

Exemplary Embodiment 50. The method of embodiment 49, wherein the pharmaceutical is an analgesic, an anti-inflammatory agent, or an antibacterial.

Exemplary Embodiment 51. The method of embodiment 41, wherein the wound comprises broken skin tissue.

Exemplary Embodiment 52. The method of embodiment 41, wherein the wound comprises broken soft tissue within a human body.

Exemplary Embodiment 53. A kit for initiating blood clotting, said kit comprising a fast-clotting wound dressing impregnated with a hemostatic agent and a phospholipid; and instructions for using the kit.

Exemplary Embodiment 54. A fast-clotting wound dressing of any of embodiments 27-36 wherein said one or more phospholipids are phosphatidylcholine, phosphatidylserine and/or sphingomyelin or derivatives thereof.

Exemplary Embodiment 55. A method of any of embodiments 37-52 wherein said one or more phospholipids are phosphatidylcholine, phosphatidylserine and/or sphingomyelin or derivatives thereof.

Exemplary Embodiment 56. A kit of embodiment 53 wherein said hemostatic agent is tissue factor and said phospholipid is phosphatidylcholine, phosphatidylserine and/or sphingomyelin or derivatives thereof.

Exemplary Embodiment 57. A fast-clotting wound dressing of embodiment 27 wherein said one or more hemostatic agents are tissue factor, chitosan, deacetylated chitin, zeolite, fibrin sealant, smectite, iron oxyacid salt, kaolin, celite, micronized silica and/or ellagic acid.

Exemplary Embodiment 58. A fast-clotting wound dressing of embodiment 57 wherein said one or more phospholipids are phosphatidylcholine, phosphatidylserine and/or sphingomyelin or derivatives thereof.

Exemplary Embodiment 59. A method of embodiment 41 wherein said one or more hemostatic agents are tissue factor, chitosan, deacetylated chitin, zeolite, fibrin sealant, smectite, iron oxyacid salt, kaolin, celite, micronized silica and/or ellagic acid.

Exemplary Embodiment 60. A method of embodiment 59 wherein said one or more phospholipids are phosphatidylcholine, phosphatidylserine and/or sphingomyelin or derivatives thereof.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

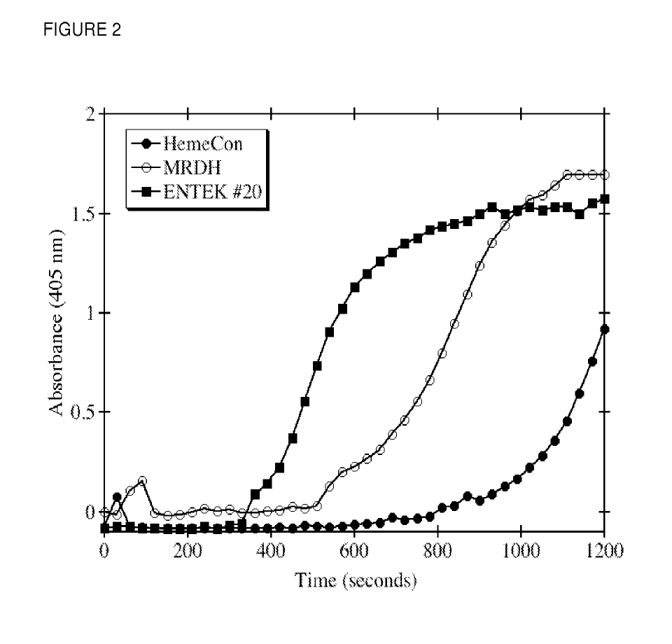

A quantitative spectrophotometric assay to assess wound dressings impregnated with coagulation initiators as described in [Englehart, J Trauma. 2008; 65:884-90], which is incorporated by reference herein for its teachings regarding the same, will be used. In this assay, a 1 cm² sample of the dressing is first placed in the bottom of a thermostated spectrophotometer cuvette at 37° C. 1 ml of standardized FACT citrated human plasma (George King Biomedical, Overland Park, Kans.) is pre-warmed to 37° C. and re-calcified to 10 mM CaCl₂, then pipetted into the cuvette with the dressing. The turbidity at 405 nm is followed spectrophotometrically for 20 minutes. Using this assay, several proprietary dressings from ENTEK, as well as dressings from HemCon and MRDH are tested. FIG. 2 shows that the currently-available dressings from HemCon and MRDH are markedly slower at initiating coagulation compared to an experimental dressing from ENTEK Manufacturing (Lebanon, Oreg.). This assay will be used to optimize the amount of hemostatic agent (e.g., tissue factor) and optionally a phospholipid in the gauze to achieve rapid coagulation of blood plasma.

### Example 2

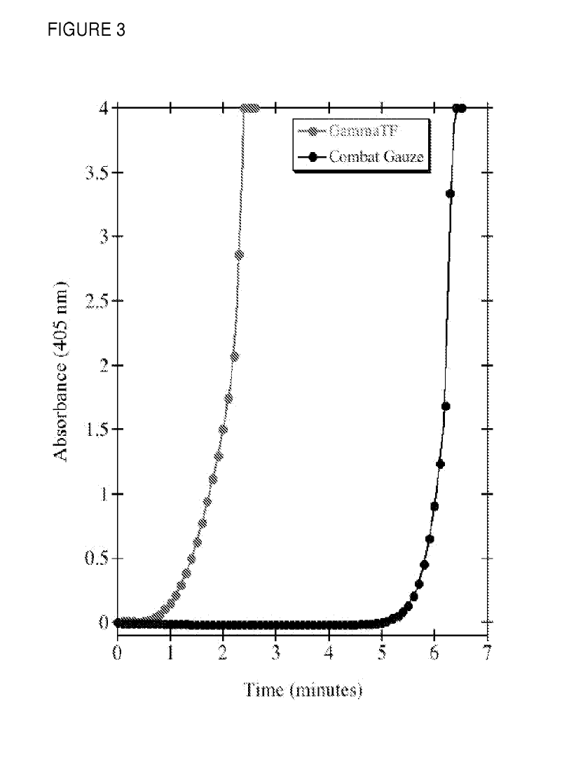

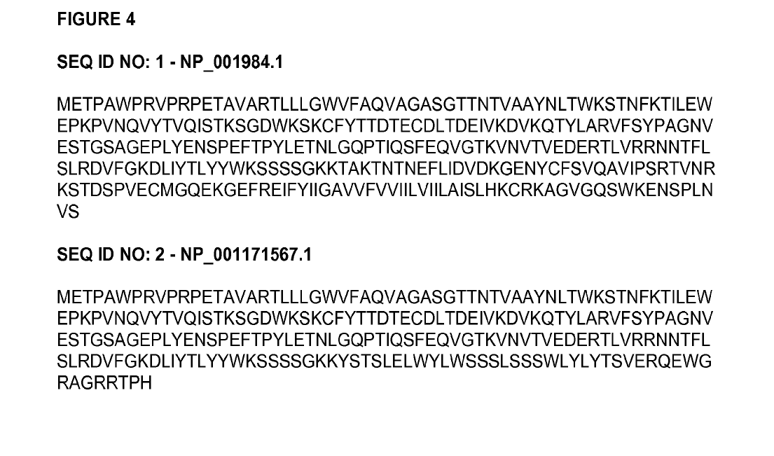

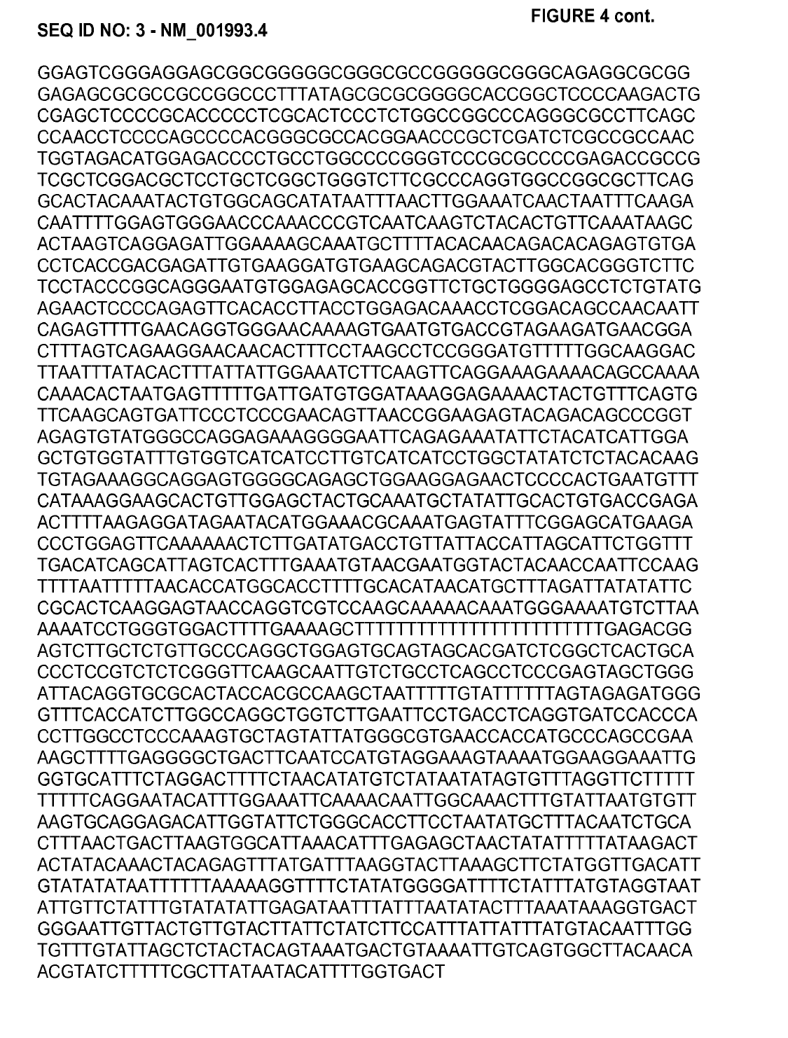

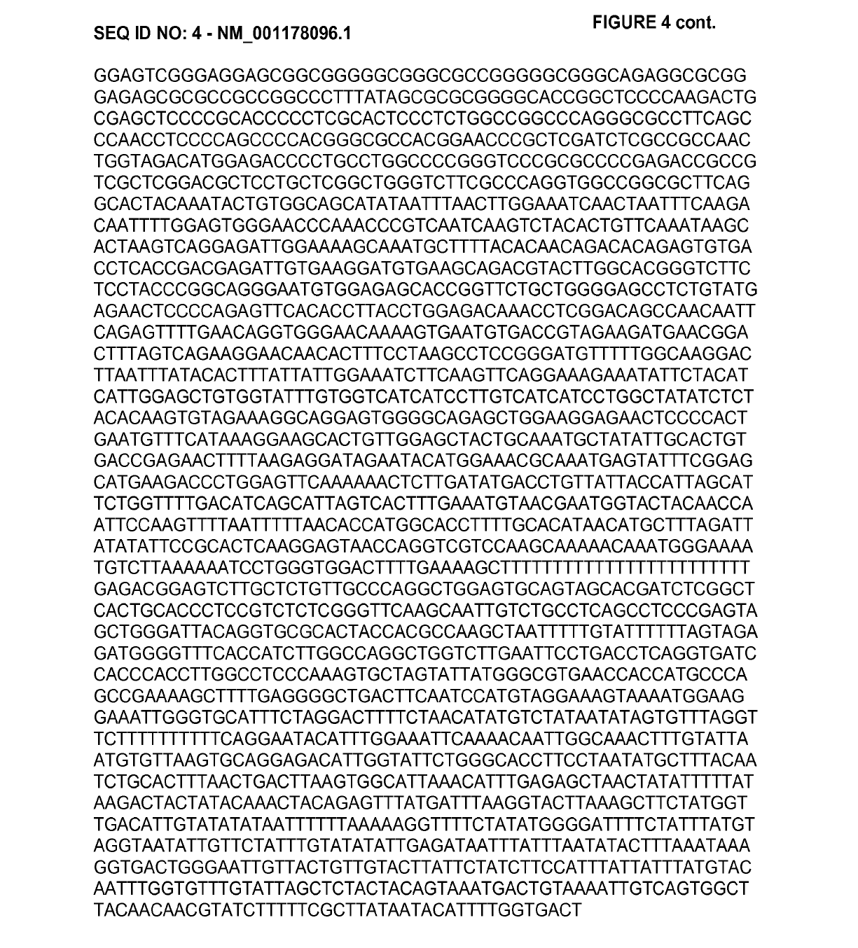

The data generated in FIG. 3 comparing clotting times using a dressing impregnated with tissue factor and phospholipid against Combat Gauze® was obtained using a slightly modified version of the assay described above. Particularly, dressings were assayed for their procoagulant activity using a spectrophotometric clotting assay. 1 cm×1 cm samples of each dressing were placed in the bottom of a spectrophotometer cuvette at 37° C. 2 ml normal human citrated plasma (FACT, George King Biomedical) were warmed to 37° C. for 5 minutes and then re-calcified with 42 μl of 1 M CaCl2. The re-calcified plasma was added to the cuvette and incubated at 37° C. The absorbance at 405 nm was measured using a kinetic program on a Shimadzu UV Mini spectrophotometer. The data demonstrates that wound dressing disclosed herein impregnated with tissue factor and phospholipid produce clotting more quickly than Combat Gauze®. The dressings were impregnated with tissue factor and phospholipid as described below.

### Example 3

Methodology—Cotton gauze dressings at standard military dimensions of 10×17.5 cm (Johnson & Johnson) will be immersed in a saline solution containing a hemostatic agent (e.g., tissue factor) at different concentrations and phospholipids at different concentrations, and the dressings will be flash frozen and lyophilized to dryness to prevent denaturation of the hemostatic agent and/or phospholipid. The saline solution will consist of 10 mM sodium phosphate, pH 7.4/150 mM NaCl/1 mM CaCl₂ and varying concentrations of synthetic phospholipids, similar to the conditions used in a prothrombin time assay. The phospholipids will be an emulsion containing synthetic phosphatidylcholine and phosphatidylserine in combination with highly purified sphingomyelin (Rossix, Molndal, Sweden). In an exemplary method, recombinant tissue factor may be obtained from Prospec (East Brunswick, N.J.). Lyophilized dressings will be stored in a desiccator until use. The dressings will then be tested using the turbidimetric assay described above. The kinetics of coagulation of the hemostatic agent-impregnated dressings impregnated with phospholipid(s), standard gauze, and Combat Gauze® will be analyzed by comparing the times to half-maximal coagulation. In this way, quantitative comparisons can be made among the different dressings, and the optimal amount of hemostatic agent and the optimal amount of phospholipid for the dressing will be determined.

1) Optimize the Amount of Hemostatic Agent or Hemostatic Agent and Phospholipid in the Gauze to Achieve Rapid Coagulation of Blood Plasma.

This will be accomplished by infusing the gauze with a range of hemostatic agent (e.g., tissue factor) concentrations in saline and lyophilizing the gauze. The gauze may optionally be infused with a range of phospholipid concentrations. Gauze samples will then be assayed for their ability to initiate coagulation in recalcified plasma in vitro. The turbidity of the resulting clot will be followed by spectrophotometry.

2) Quantitate the Effect of the Hemostatic Agent Impregnated Gauze or the Hemostatic Agent and Phospholipid Impregnated Gauze on Blood Loss In Vivo.

Preliminary Studies—The swine injury model described in [Englehart, J Trauma. 2008; 65:884-90; Sambasivan, Am J Surg. 2009; 197:576-80], which are incorporated by reference herein for their teachings regarding the same will be utilized. Briefly, the model involves the use of a standardized 8 cm incision that exposes the femoral artery, followed by a 6 mm punch biopsy directly into the femoral artery.

Methodology—Swine Severe Groin Injury Model: Yorkshire crossbred pigs weighing approximately 30-40 kg will be fasted for 16 hours the day before surgery. Water will be available ad libitum. A randomized controlled trial comparing Combat Gauze® to gauze impregnated with a hemostatic agent to gauze impregnated with a hemostatic agent and a phospholipid to standard gauze for hemorrhage control of a severe groin injury will be tested.

On the day of the experiment animals will be given an induction agent consisting of Telazol® (Wyeth, LLC; Madison, N.J.), 8 mg/kg given intramuscularly. A 7.5 mm internal diameter cuffed endotracheal tube will be placed. Animals will be orotracheally intubated and mechanically ventilated keeping pCO₂ values between 40-45 torr. They will be monitored via an esophageal thermometer and kept normothermic (38.0±1.5° C.) using external warming devices. The endotracheal tube will be connected to the anesthesia machine with 1-3% isoflurane for anesthetic maintenance in 50% oxygen. Tidal volume will be fixed at 7-12 ml/kg with a rate of 10 breaths per minute. Ventilation will be adjusted to maintain a PCO₂ around 40 mm Hg based on the baseline arterial blood gas. An esophageal thermometer will be inserted.

Animals will be placed in the supine position and the ventral cervical area, ventral abdomen and hair from the left femoral area will be clipped. A left ventral cervical cutdown will be made and a 1.5 mm catheter will be placed in the carotid artery for continuous monitoring of blood pressures and blood collection. In addition, a 1.5 mm catheter will be placed in the external jugular vein. The venous catheter will be used for administration of resuscitation fluid. Mean arterial pressure (MAP) and heart rate (HR) will be continuously recorded and averaged every 10 seconds using a digital data collection system with a blood pressure analyzer (DigiMed, Louisville, Ky.). The animals will undergo a lower midline celiotomy and suprapubic Foley catheter placement. The abdomen will then be closed with towel clamps. A standardized 8 cm incision will be created in the groin to expose the femoral artery.

Baseline MAP will be recorded and a femoral arteriotomy created using a 6 mm punch biopsy instrument. This will be followed by 30 seconds of uncontrolled hemorrhage that will be collected and recorded. Animals will be randomized to receive one of the 4 aforementioned dressings. Following uncontrolled hemorrhage, the randomized dressing will be applied through a small pool of blood into the wound and pressure held for up to 5 minutes. The exact time of the compression period will be determined during the model development phase. The team member performing the dissection will be blinded to the dressing type until the time of application. The team member applying pressure will also be blinded to the dressing type by covering the dressings with a laparotomy sponge.

After the compression period, pressure will be released and fluid resuscitation initiated. Animals will be monitored for 120 minutes and surviving animals sacrificed. Following completion of the study, the animals will be euthanized with Beuthanasia solution (1 ml/10 lb). The wound will be inspected for bleeding with failure of hemostasis defined as blood pooling outside of the wound. Blood loss is calculated as the sum of extravasated blood suctioned into pre-weighed suction canisters and blood collected in pre-weighed dressings.

Post-mortem, all wounds will be inspected to ensure a similar injury pattern and proper dressing application. Labs (ABG, hematocrit, and TEG) will be obtained at baseline, 60 minutes, and 120 minutes following injury. Baseline demographics such as weight, pre-injury MAP, and pre-treatment blood loss, as well as post-compression blood loss, total study blood loss, urine output, dressing success, and mortality will be followed and recorded.

Sample Size Justification: Based on previous studies of hemostatic dressings, it is estimated that the mean difference in post-compression blood loss between the new hemostatic dressings and standard gauze will be 60%, with the hemostatic dressing groups experiencing 60% less blood loss than that seen in the gauze group, with a standard deviation difference of 56%. Using a power of 90% and a P value of 0.05, it is estimated that 12 animals per group will be required to show changes in dressing effectiveness. This predicted sample size was calculated using SamplePower® Version 2.0 (SPSS Inc., Chicago, Ill.).

Results of these studies demonstrate that the tissue factor-impregnated wound dressings disclosed herein initiate coagulation faster than Combat Gauze® in vitro. Further, these results also demonstrate that the tissue factor and phospholipid impregnated wound dressings disclosed herein initiate coagulation faster than wound dressings without a phospholipid co-factor disclosed herein. Additional studies will demonstrate that the wound dressings disclosed herein result in decreased blood loss in a swine injury model compared to Combat Gauze® in vivo.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

What is claimed is:

1. A fast-clotting wound dressing impregnated with tissue factor and/or analogues, homologues, precursors or derivatives thereof.

2. The fast-clotting wound dressing of claim 1, further comprising one or more phospholipids impregnated within said dressing.

3. The fast-clotting wound dressing of claim 2 wherein said one or more phospholipids are phosphatidylcholine, phosphatidylserine and/or sphingomyelin.

4. The fast-clotting wound dressing of claim 1, wherein the tissue factor is human tissue factor.

5. The fast-clotting wound dressing of claim 4, wherein the human tissue factor is produced using recombinant technology.

6. The fast-clotting wound dressing of claim 1, wherein said wound dressing initiates clotting within 120 seconds of application to a wound.

7. The fast-clotting wound dressing of claim 1, wherein said wound dressing initiates clotting within 27 seconds of application to a wound.

8. The fast-clotting wound dressing of claim 1, wherein tissue factor is impregnated at a concentration of 5 pmoles-100 pmoles per bandage.

9. The fast-clotting wound dressing of claim 1, wherein the dressing is further impregnated with a secondary clotting factor.

10. The fast-clotting wound dressing of claim 1, wherein the dressing is further impregnated with one or more secondary agents selected from the group consisting of pharmaceuticals, antifungal agents, topical pain reducing medication, pharmaceuticals, anti-inflammatory agents and tissue enzyme inhibitors.

11. The fast-clotting wound dressing of claim 10, wherein the pharmaceutical is an analgesic, an anti-inflammatory agent, or an antibacterial.

12. A fast-clotting wound dressing impregnated with one or more hemostatic agents and one or more phospholipids.

13. The fast-clotting wound dressing of claim 12, wherein the hemostatic agent is tissue factor and/or analogues, homologues, precursors or derivatives thereof.

14. The fast-clotting wound dressing of claim 13, wherein the tissue factor is human tissue factor.

15. The fast-clotting wound dressing of claim 14, wherein the human tissue factor is produced using recombinant technology.

16. The fast-clotting wound dressing of claim 12, wherein said wound dressing initiates clotting within 27 seconds of application to a wound.

17. The fast-clotting wound dressing of claim 12, wherein the hemostatic agent is impregnated at a concentration of 5 pmoles-100 pmoles per bandage.

18. The fast-clotting wound dressing of claim 12, wherein the dressing is further impregnated with an additional clotting factor.

19. The fast-clotting wound dressing of claim 12, wherein the dressing is further impregnated with one or more agents selected from the group consisting of pharmaceuticals, antifungal agents, topical pain reducing medication, pharmaceuticals, anti-inflammatory agents and tissue enzyme inhibitors.


SEQUENCE LISTING (4 sequences)

SEQ ID NO: 1 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 2 - protein, 238 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKYSTSLELWYLWSSSLSSSWLYLYTSVERQEWGRAGRRTPH

SEQ ID NO: 3 - DNA, 2393 bp, Homo sapiens
ggagtcgggaggagcggcgggggcgggcgccgggggcgggcagaggcgcgggagagcgcgccgccggccctttatagcgcgcggggcaccggctccccaagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacgaatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagcttttttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacgccaagctaatttttgtattttttagtagagatggggtttcaccatcttggccaggctggtcttgaattcctgacctcaggtgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgttactgttgtacttattctatcttccatttattatttatgtacaatttggtgtttgtattagctctactacagtaaatgactgtaaaattgtcagtggcttacaacaacgtatctttttcgcttataatacattttggtgact

SEQ ID NO: 4 - DNA, 2233 bp, Homo sapiens
ggagtcgggaggagcggcgggggcgggcgccgggggcgggcagaggcgcgggagagcgcgccgccggccctttatagcgcgcggggcaccggctccccaagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacgaatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagcttttttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacgccaagctaatttttgtattttttagtagagatggggtttcaccatcttggccaggctggtcttgaattcctgacctcaggtgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgttactgttgtacttattctatcttccatttattatttatgtacaatttggtgtttgtattagctctactacagtaaatgactgtaaaattgtcagtggcttacaacaacgtatctttttcgcttataatacattttggtgact